(19) United States
(12) Patent Application Publication
(10) Pub. No.: US 2011/0229918 A1
(43) Pub. Date: Sep. 22, 2011

(54) Method of Quantifying Transient Interactions Between Proteins

(75) Inventors: Nicole Schneider-Han-Marra, Wannweil (DE); Andreas Brecht, Muttenz (CH)

(73) Assignee: COVALYS BIOSCIENCES AG, Witterswil (CH)

(21) Appl. No.: 12/742,198

(22) PCT Filed: Nov. 7, 2008
(86) PCT No.: PCT/EP2009/065129

§ 371 Date: Jun. 7, 2011

(30) Foreign Application Priority Data
Dec. 11, 2008 (DE) 102008062614.7

(51) Int. Cl. C12Q 1/34; G01N 33/68; C12Q 1/02; C07H 19/207; C07D 205/00; C07D 239/47; C07D 403/02; C07D 239/00; C07D 473/18

(52) U.S. Cl. 435/18; 436/86; 435/29; 536/26.23; 540/200; 544/317; 544/296; 544/242; 544/276

(57) ABSTRACT

The invention relates to a precursor for producing sintered metallic components, a method for producing the precursor and the production of the components. The object of the invention is to disclose possibilities of being able to produce sintered metallic components, which render possible an increased physical density and a reduced shrinkage on the fully sintered component. With a precursor according to the invention for the production of sintered metallic components, a coating layer is formed on a core, which is formed from respectively one particle of a first metallic powder. The coating layer is formed with a second powder and a binder. The first powder thereby has a particle size d₉₀ of at least 50 μm and the second powder has a particle size d₉₀ of less than 25 μm. The precursor is powdery.

The invention relates to a precursor for producing sintered metallic components, a method for producing the precursor and the production of the components.

For the production of sintered metallic components, powders are used, these are usually made from the respective metal and as a rule from the metal alloy with which the component is to be produced. For the production of the components, a crucial influence can be achieved through the selection or pretreatment of the initial powder, which determine the properties of the component. Thus the particle size of the powder used has a strong influence on the physical density of the component material that can be achieved and the shrinkage during sintering.

In the past, the sintering activity could be improved in particular by a high-energy milling carried out in advance and the properties of the component material could also be improved thereby.

Other demands are also made on the metal powder used. For a processing in the production of greenbodies, a good flowability of the powder, an increased green density and green strength of the greenbodies before sintering are desirable. If during the shaping by pressing higher green densities of the greenbodies are achieved, the shrinkage occurring on the fully sintered component is reduced. However, a very small shrinkage is desirable in order to be able to produce strongly contoured components and not to have to carry out a finishing treatment.

High-alloy metallic powders cannot be processed to form sintered components by simple powder metallurgical technologies, such as pressing and sintering, due to the hardness present. Through a high-energy milling of such alloyed powders and subsequent agglomeration, powders of this type are, e.g., injectable. However, with the increased sintering activity, poorer technological parameters, such a low packing density, poor flow behavior and a high shrinkage during sintering have to be accepted. Due to these disadvantageous properties, it is not possible to produce high-density components without considerable mechanical finishing.

Sintered components produced in a conventional manner achieve physical densities that are about 95% of the theoretical density and have a shrinkage of at least 10%.

The object of the invention is therefore to disclose possibilities of being able to produce sintered metallic components, which render possible an increased physical density and a reduced shrinkage on the fully sintered component.

According to the invention, this object is attained with a precursor that has the features of claim 1. It can be produced with a method according to claim 7. Claim 11 relates to the production of sintered metallic components. Advantageous embodiments and further developments of the invention can be achieved with features described in subordinate claims.

The invention is directed at advantageous possibilities for producing sintered metallic components. A powdery precursor is thereby used, which is subjected to a shaping and sintering in place of the metal powder previously used.

The precursor is composed of cores that are enclosed by a coating layer. For the production, a first and a second powder are used, which differ at least in their particle size. Thus the particles of the first powder, which form cores, are larger and have a particle size d₉₀ of at least 50 μm, preferably at least 80 μm. It is a metal or a metal alloy.

The particles of the second powder are smaller and have a particle size d₉₀ less than 25 μm, preferably less than 20 μm and very particularly preferably they are smaller than 10 μm. In addition, the coating layer contains a binder. This can preferably be organic. For example, polyvinyl alcohol (PVA) can be used as a binder. The second powder can be a metal, a metal alloy or a metal oxide. However, it can also be a mixture with at least two of these components. In addition, carbon can be contained in the form of graphite.

In the simplest case, the particles of the first and the second powder can be formed of the same metal or the same metal alloy. However, it is advantageous to use different metals, metal alloys for the two powders or also to use a metal oxide for the second powder. This makes it possible during sintering, which is carried out to produce a finished component, to also achieve at the same time an alloy formation or through an equalization of concentration of alloying constituents a changed alloy composition on the finished component material.

It is favorable for the further processing in the production of greenbodies and the finished components, if the second powder is more ductile than the first powder. During pressing for the production of greenbodies a higher green density can thereby be achieved with a shaping process, which ultimately also leads to a higher physical density of the component after sintering and to a lower shrinkage. The coating layer thereby performs a function that is to be assessed as analogous to that of pressing aids.

With a precursor, the individual particles of the precursor should be produced such that the coating layer has a weight percentage that is no greater than the weight percentage of a core. The proportion of binder in the coating layer can thereby be disregarded or negligible. However, the weight percentage of the cores should preferably be greater than that of coating layers. Coating layers should also have the same layer thicknesses, which should apply to the individual and also to all particles of the precursor.

The precursors according to the invention can be produced by spraying the particles of the first powder with a suspension. The suspension thereby contains particles of the second powder and the binder. An aqueous suspension can be used. During spraying, the particles of the first powder are moved. For this purpose, for example, a fluid bed rotor can be used.

After a predetermined layer thickness of the coating layers has been achieved on the particles of the first powder forming cores, the particles of the precursor can be dried. A high packing density of approx. 40% of the theoretical density and a good flowability can thus be achieved, which can be less than 30 s, which is determined with a Hall Flowmeter funnel.

In addition, a presintering of the precursor can be carried out. Further influence on the properties of the precursor in terms of its packing density and flowability can thereby be achieved. The packing density can be increased and the flowability can be improved thereby. The latter can be thus reduced, e.g., from 40 s to 30 s, if a presintering at a temperature of at least 800° C. is carried out. It can be determined thereby with a Hall

Flowmeter funnel. The physical density of the fully sintered component can thus be increased and the shrinkage also reduced to less than 5%.

The precursor can then be subjected to a shaping. Compacting forces thereby act, which lead to a compacting. The greenbodies obtained thereby achieve an increased green density and green strength. During the pressing, essentially the components contained in the coating layer are deformed. The cores thereby generally remain undeformed. Through the deformation of the coating layer an increased compacting can be achieved, with leads to a reduction of shrinking during sintering. This can be kept to less than 8%. A reduction to 5% and lower is also possible. The physical density of a fully sintered component can reach at least 92% and up to or above 95% of the theoretical density.

As already mentioned, during sintering an alloy formation or a changed alloy composition can take place. An equalization of concentration thereby takes place between the two powders used for the cores and the coating layer when they have a consistency or composition deviating from one another. Diffusion processes can be utilized. The longest diffusion path is thereby 0.5 times the precursor particle diameter. The time necessary for a diffusion can be clearly reduced compared to conventional production methods. This also applies compared to the known use of diffusion-bonded powders, in which, e.g., particles of nickel or molybdenum are sintered to particles of pure iron. However, only a very small proportion of alloying elements, which is in the range of 0.1 to 2%, can be achieved thereby. In contrast, with the invention much higher alloyed component materials can be obtained. The consistency of an alloy that can be produced using the invention by sintering can be adjusted very precisely and manufactured in a reproducible manner compared to the known technical solutions.

Thus different iron-base, cobalt-base and also nickel-base alloys can be produced. The proportion of the respective base metal is thereby at least 50% by weight.

The invention is described in more detail below based on examples.

### EXAMPLE 1

A component is to be produced thereby in which the component material is a 5.8 W, 5.0 Mo, 4.2 Cr, 4.1 V, 0.3 Mn, 0,3 Si, 1.3 C iron alloy.

For the first powder forming the cores of the precursor, an iron base alloy with 8.1 W, 6.7 Mo, 5.9 Cr, 0.4 Mn, 0.4 Si is used. The particle size d₉₀ was thereby 95 μm.

For the coating layer a second powder was used, which represents a mixture of 31.0% by weight carbonyl iron powder and 1.3% by weight partially amorphous graphite with respectively a particle size d₉₀ of less than 10 μm. This resulted in a weight percentage for the cores of 67.7% by weight and 32.3% by weight coating layer without binder.

The carbonyl iron was reduced, but it can also be used unreduced.

The first powder was placed as the initial charge into a fluid bed rotor and moved thereby. A suspension that had been formed with water, PVA and the powder mixture for the coating layer was sprayed through a two-fluid nozzle arranged tangentially to the direction of rotation of the rotor. The buildup of the coating layer around the cores should take place as slowly as possible. The composition of the suspension was 38% by weight water, 58% by weight carbonyl iron powder, 2.4% by weight partially amorphous graphite and 1.8% by weight binder (PVA).

After a drying, the powdery precursor product had a particle size d₉₀ of 125 μm.

Subsequently, a shaping for a pressing for the compacting and the embodiment of a greenbody was carried out. For this purpose, the usual shaping methods can be used, such as for example a matrix pressing in molds, injection molding or extrusion. It was possible to achieve a green density of 6.9 g/cm³ and a green strength of 10.3 MPa.

Thereafter the greenbody was sintered under formier gas (10% by volume H₂ and 90% by volume N₂). The heat treatment was carried out in stages at 250° C., 350° C. and 600° C. with 0.5 h retention time in each case. The maximum temperature of 1200° C. was held over 2 h.

The fully sintered component had a physical density of 7.95 g/cm³ and the shrinkage after the sintering was 4.6%. The theoretical density of this material is 7.97 g/cm³.

### EXAMPLE 2

For the production of a component from an iron base alloy 34.0 Cr, 2.1 Mo, 2.0 Si, 1.3 C the rest being iron, a first powder was used for the cores with an alloy 51.5 Cr, 3.6 Mo, 2.7 Si, 0.68 Mn, 1.9 C, the rest being iron with a particle size d₉₀ of 82 μm.

For the second powder, as variant 1 unreduced carbonyl iron powder (particle size d₉₀ 9 μm) was used and as variant 2 iron powder was used that has been obtained from reduced iron oxide (particle size d₉₀ 5 μm).

For the first powder, the weight percentage was 66.7% and for the second powder respectively 33.3% by weight.

The first powder was placed as the initial charge in a fluid bed rotor and moved thereby. A suspension that had been formed with water, PVA and the powder mixture for the coating layer was sprayed through a two-fluid nozzle arranged tangentially to the direction of rotation of the rotor. The buildup of the coating layer around the cores should be carried out as slowly as possible. The suspension had a composition of 49% by weight water, 49% by weight of the second powder and 2% by weight binder (PVA).

The precursor according to variant 1 had a packing density of 2.2 g/cm³ with a flow time determined by a Hall Flowmeter funnel of 36 s. For the precursor according to variant 2, it was possible to achieve a packing density of 2.4 g/cm³ and a flow time of 33 s was determined.

Subsequently, a shaping for a pressing for the compacting and the embodiment of a greenbody was carried out. For this purpose the usual shaping methods can be used, such as for example a matrix pressing in molds, injection molding or extrusion.

A greenbody according to variant 1 achieved a green density 5.3 g/cm³ and a green strength of 3.8 MPa and for variant it was possible to achieve a green density of 5.4 g/cm³ and a green strength of 5.0 MPa.

Thereafter the greenbody with both variants was sintered under formier gas (10% by volume H₂ and 90% by volume N₂). Thereby a temperature regime in steps of respectively 0.5 h retention time at temperatures of 250° C., 350° C. and 600° C. was maintained. Subsequently, at 1250° C. sintering was completed for a period of 2 h.

The fully sintered component for variant 1 had a physical density of 7.1 g/cm³ and the shrinkage after sintering was 7.6%, and for variant 2 a physical density of 6.9 g/cm³ and a shrinkage of 6.3% occurred. The theoretical density of this material is 7.35 g/cm³.

### EXAMPLE 3

For the production of a component with a target alloy as a cobalt base alloy with the composition of 27.6 Mo, 8.9 Cr, 2.2 Si, the rest being cobalt, a first water-atomized powder of an alloy of 27.6 Mo, 8.9 Cr, 2,2 Si, the rest being cobalt with a particle size d₉₀ of 53.6 μm and a second powder of an alloy of 27.6 Mo, 8.9 Cr, 2.2 Si the rest being cobalt with a particle size d₉₀ of 21 μm was used. Both powders were used for the production of the precursor with respectively 50% by weight. The suspension had a composition of 29% by weight water, 69% by weight of the second powder, 1% by weight paraffin and 1.4% by weight binder (PVA).

The first powder was placed as an initial charge into a fluid bed rotor and moved thereby. A suspension that was formed with water, PVA and the powder mixture for the coating layer was sprayed through a two-fluid nozzle arranged tangentially to the direction of rotation of the rotor. The buildup of the coating layer around the cores should take place as slowly as possible.

After a drying, the powdery precursor had a particle size d₉₀ of 130 μm. The packing density was 3.0 g/cm³ and it was possible to determine a flow time of 29 s with a Hall Flowmeter funnel.

Subsequently, a shaping for a pressing for the compacting and the embodiment of a greenbody was carried out. For this purpose the usual shaping methods can be used, such as for example a matrix pressing in molds, injection molding or extrusion. A green density of 6.4 g/cm³ was achieved.

Thereafter the greenbody was sintered with the following parameters in a hydrogen atmosphere:

A heat treatment in stages at temperatures of 250° C., 350° C. and 600° C. respectively with a retention time of 0.5 h and subsequently an increase of the temperature to 1285° C. was carried out. The maximum temperature was maintained over 2 h.

The fully sintered component had a physical density of 8.7 g/cm³ and the shrinkage after sintering was 10.2%.

1-15. (canceled)

16. A method of detecting and quantifying a transient interaction between a first and a second protein, comprising:
fusing the first protein to a binder protein to form a fusion protein;
linking the second protein to a substrate which is specific for the binder protein to form a substrate protein;
interacting the fusion protein with the substrate protein to form a reaction product;
and detecting and quantifying a transient interaction between the first and the second protein.

17. The method of claim 16, wherein the transient interaction between the two different proteins is indirect.

18. A method according to claim 17, further comprising adding at least a third protein for generating a multi-protein interaction.

19. The method according to claim 16, further comprising expressing the fusion protein in a host cell as a recombinant protein.

20. The method according to claim 16, wherein the binder protein is selected from the group consisting of AGT, ACT, Halotag, serine-beta-lactamases, and Acyl Carrier Proteins and modifications thereof.

21. The method according to claim 16, wherein the substrate is selected from the group consisting of benzylguanine derivatives, pyrimidine derivatives, benzylcytosine derivatives, chloroalkane derivatives, beta-lactam derivatives and Coenzyme A derivatives.

22. The method of claim 16, wherein the substrate protein further comprises an affinity tag bound to the substrate.

23. The method according to claim 22, further comprising an affinity tag binding protein bound capable of binding to the affinity tag bound to the substrate.

24. The method of claim 16, further comprising reacting the substrate protein with the fusion protein to form a covalent linkage with one substrate subunit of a bifunctional substrate.

25. The method according to claim 16, further comprising interacting the fusion protein and substrate protein only when a target protein is present, for detecting and quantifying an interaction between the first protein, the second protein and the target protein.

26. The method according to claim 16, further comprising adding substrate in an effective amount to inhibit the interaction between the fusion protein and the substrate protein.

27. The method of claim 16, wherein the transient interaction is dependent on phosphorylation of the first or second protein.

28. The method of claim 16, wherein the transient interaction is dependent on dephosphorylation of the first or second proteins.

29. The method of claim 16, wherein at least one of the first or second protein is a small GTPase activated by GTP binding and the other protein is a protein binding domain recognized by the activated GTPase.

30. The method according to claim 16, wherein the substrate comprises two identical or different substrate subunits independently selected from pyrimidine derivatives, benzylcytosine derivatives, chloroalkane derivatives, beta-lactam derivatives, and Coenzyme A derivatives, optionally connected through a linker.

31. The method according to claim 16, wherein the substrate comprises a benzylguanine and a second substrate subunit selected from pyrimidine derivatives, benzylcytosine derivatives, chloroalkane derivatives, beta-lactam derivatives, and Coenzyme A derivatives, optionally connected through a linker.

32. An assay kit for the detection and quantification of transient protein interactions according to the method of claim 16.

33. A substrate comprising two identical or different substrate subunits independently selected from pyrimidine derivatives, benzylcytosine derivatives, chloroalkane derivatives, beta-lactam derivatives, and Coenzyme A derivatives, optionally connected through a linker.

34. A substrate comprising a benzylguanine and a second substrate subunit selected from pyrimidine derivatives, benzylcytosine derivatives, chloroalkane derivatives, beta-lactam derivatives, and Coenzyme A derivatives, optionally connected through a linker.
